(19) United States
(12) Patent Application Publication
(10) Pub. No.: US 2017/0369211 A1
(43) Pub. Date: Dec. 28, 2017

(54) TAMPER EVIDENT SECURITY LID FOR A MEDICAL CONTAINER

(71) Applicant: Killara I.P. Pty Ltd, Bayswater, Victoria (AU)

(72) Inventor: Mark Verschuur, Bayswater, Victoria (AU)

(21) Appl. No.: 15/540,526

(22) PCT Filed: Feb. 4, 2016
(86) PCT No.: PCT/AU2016/050062

§ 371 Date: Jun. 28, 2017

(30) Foreign Application Priority Data
Feb. 4, 2015 (AU) 2015900338

(51) Int. Cl. B65D 41/34; A61B 50/30; A61B 50/00

(52) U.S. Cl. CPC B65D 41/3404; A61B 50/30; A61B 2050/005

(57) ABSTRACT

A lid for a medical container including a body portion including a locating element, a tamper evident security connector integrally formed with the body portion, the tamper evident security connector including an anchor portion and a frangible portion. The anchor portion can attach to and be retained by an engagement portion fixed in at least one direction relative to the medical container, thereby positioning the frangible portion between the locating element and engagement portion. The frangible portion is adapted to break on relative movement between the lid and the medical container. The lid can be suitable for use in an autoclave, and can be formed from a material or materials that are capable of withstanding a temperature of at least 140° C.

### BACKGROUND

### Technical Field

The present invention relates to a lid for a medical container, the lid including a tamper evident security connector.

### Description of the Related Art

Medical containers may be used to store medical equipment, medicines, or other biological materials. Depending on their intended use, medical containers may also be used to treat the contents that are retained therein, for example the container may be used for sterilizing medical equipment in an autoclave or by chemical treatment.

The containers may be provided in a two-piece form, e.g., including a container portion such as a bowl, tray, tub or other storage element and a top lid. Alternatively, these containers may be provided in a one-piece form wherein the container portion and lid are connected via, for example, a hinge. These types of containers are typically made by thermoforming or blow molding plastics, such as polyesters, polyethylene terephthalate (PETE), polylactic acid (PLA), polyvinyl chloride (PVC), polystyrene (PS), polypropylene (PP) and the like.

A range of different closure mechanisms are possible, depending on whether the container is provided in a two-piece form, or a one-piece form. Such closure mechanism may include use of a screw cap, snap on cap, friction fit cap, or a variety of other closure mechanisms which are known to those skilled in the art.

Because these containers typically contain items such as medicines, sterilized equipment, or biological materials, attempts have been made to provide a tamper evident lid so that a user can determine or detect when the container has been opened or otherwise tampered with. However, there is a need to provide new tamper evident security connectors that can be used with medical containers. For example, it is desirable to provide tamper evident lids that are inexpensive and easy to produce, as well as being easy and convenient for use by the skilled addressee.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### BRIEF SUMMARY

In one aspect of the invention there is provided a lid for a medical container, the lid including:
a body portion including a locating element,
a tamper evident security connector integrally formed with the body portion, the tamper evident security connector including:
   an anchor portion, and
   a frangible portion;
wherein the anchor portion is configured to attach to and be retained by an engagement portion fixed in at least one direction relative to the medical container, thereby positioning the frangible portion between the locating element and engagement portion, the frangible portion being adapted to break on relative movement between the lid and the medical container; and
wherein the lid is suitable for use in an autoclave, the lid being formed from a material or materials that are capable of withstanding a temperature of at least 140° C.

The invention is particularly applicable to medical sterilization containers, such as medical containers for sterilizing an object and storing the sterilized object therein. It is preferred that the container and lid, when assembled, are permeable to sterilization agent to allow the passage of the sterilization agent into and out of the assembled container, so that the sterilization agent can contact an instrument held therein. Thus, in one embodiment, the lid further includes a permeable portion that allows transmission of a sterilization agent (preferably steam).

The skilled addressee is readily able to assess whether a container having the lid attached has been opened or tampered with by assessing whether the frangible portion has been broken or not. Furthermore, in such instances, the skilled addressee may also inspect the engagement portion of the container to ascertain whether the anchor portion of the broken tamper evident security connector has been retained therein.

Preferably the body portion and the tamper evident security connector are unitary. The term “unitary,” as used here, is intended to define that these constituent components of the lid are a single unit.

In an embodiment, the engagement portion is fixed to the container collar. In an alternative embodiment, the engagement portion is part of a collar that is attached to the container, for example to a neck of the container. In such instances, the collar may be rotatable about the neck of the collar in one direction but not in the other. For example, if the lid is connectable to the container via a right-handed thread, then the collar may be rotatable about the container in the clockwise direction but not in the counter-clockwise direction. Thus, the engagement portion, attached to the collar, is fixed relative to the container in the counter-clockwise direction. A suitable mechanism to permit rotation in one direction and not the other may be through use of ratchet arrangement between the collar and the container. In still further embodiments, the medical container includes the engagement portion as an integrally formed component.

As discussed above, the lid is suitable for sterilization procedures, such as in an autoclave. In one or more embodiments, the container is adapted to receive a medical instrument therein so that the medical instrument can be sterilized in the autoclave. The lid is formed from a material or materials that are capable of withstanding a temperature of at least 140° C., preferably at least 150° C., and more preferably at least 160° C. It is further preferred that the lid is formed from a material or materials that is capable of withstanding pressures of up to 100 kPa gauge, such as usual in an autoclave. By withstanding a temperature and/or pressure it is meant that the material does not deform, degrade, or melt at that temperature and/or pressure. This is particularly preferable for situations in which the lid is used to seal a container that used for sterilization of medical equipment. In such cases, the lid may additionally include other components such as permeable portion, such as a vapor permeable membrane, which permits the passage of steam into and out of the container. The vapor permeable membrane permits the passage of vapor, which is preferably steam, into and out of the internal cavity to sterilize the medical instrument. Preferably the vapor permeable membrane is hydrophobic. The hydrophobic nature of the membrane prevents surfaces of the membrane from being wetted by water, thus avoiding strikethrough and potentially allowing pathogens to pass through the membrane. It is preferred that the vapor permeable membrane is formed from a material selected from the group consisting of polypropylene, polyester and polytetrafluoroethylene.

Preferably the anchor portion is shaped in such a manner as to allow insertion of the anchor portion into the engagement portion and to prevent withdrawal of the anchor portion from the receiving portion after insertion. That is, the anchor portion is easily insertable into the engagement portion, but retention means within the engagement portion prevents the anchor portion from being withdrawn. The retention means ideally resists an applied force to remove the anchor portion that is greater than the force required to break the frangible portion. That is, the frangible portion is configured to break on application of a separation force that is less than the force required to separate the anchor portion from the engagement portion. In this way, attempting to remove the anchor portion, for example by pulling the anchor portion from the engagement portion causes the frangible portion to break rather than separation of the anchor portion and the engagement portion.

It is preferred that the anchor portion is immediately adjacent to the frangible portion. This is so that a pulling force cannot be applied between the anchor portion and the frangible portion allowing the pulling force to by-pass the frangible portion. If frangible portion is immediately adjacent to the anchor portion, then any pulling force or tension applied to remove the anchor portion from the engagement portion will also be applied across the frangible portion.

In an embodiment, the locating element is configured to locate with the engagement portion when the lid is attached to the medical container. Preferably, the locating element is configured to coaxially align with the engagement portion.

In an embodiment, the locating element is configured to guide the anchor portion to the engagement portion.

In an embodiment the locating element is an eyelet configured to allow passage of the anchor portion therethrough to attach and be retained by the engagement portion of the medical container.

Preferably the eyelet is a plastic ring that is integrally formed with the lid body, such as an outer perimeter of the lid body wherein the outer perimeter of the lid is in a plane that is parallel with an opening of the container. More preferably, the eyelet is configured to frictionally engage with the tamper evident security connector, and to substantially retain the tamper evident security connector in position relative to the body portion of the lid.

Preferably the eyelet has an opening which is in a first plane and the engagement portion has an opening which is in a second plane, the first and second planes being substantially parallel.

Preferably, the eyelet is configured to apply a separation force to the tamper evident security connector to break the frangible portion. For example, relative movement between the lid and the medical container causes the eyelet to apply the separation force to the tamper evident security connector to break the frangible portion. For example, for a screw-threaded lid, the act of turning the lid to de-thread the lid from the container causes the eyelet to press against a portion of the tamper evident security connector causing the frangible portion to break. Similarly, the removal process for other types of lids causes the eyelet to apply interact with the tamper evident security connector to break the frangible portion.

Preferably, the eyelet is configured to coaxially align with the engagement portion so that the tamper evident security connector can be fed through the eyelet and into the engagement portion along a common shared axis.

Preferably the eyelet has an eyelet opening with an eyelet axis passing through the center thereof, the eyelet opening being in a first plane, wherein the eyelet axis is perpendicular to the first plane; and the engagement portion has an engagement opening with an engagement axis passing through the center thereof, said engagement opening being in a second plane, wherein the engagement axis is perpendicular to the first plane; and the eyelet axis and the engagement axis are coaxial when the lid is in its connected state with the container. More preferably, the eyelet axis and the engagement axis are substantially aligned.

This alignment between the openings of the eyelet and the engagement portion is advantageous as it allows the tamper evident security connector to be fed through the eyelet opening and into the engagement portion. More preferably, the frangible portion is configured to rest in a space between the eyelet and the engagement portion after attachment and retention of the anchor portion by the engagement portion. Preferably, the space between the eyelet and the engagement portion is less than about 1 cm. More preferably, less than about 0.8 cm. Most preferably, less than about 0.6 cm.

Preferably, the eyelet is configured to frictionally engage with the tamper evident security connector, and to substantially retain the tamper evident security connector in position relative to the body portion of the lid after passage of the anchor portion therethrough. More preferably the diameter of an opening in the eyelet is sufficiently large to permit passage of the tamper evident security connector, but sufficiently narrow to frictionally engage with at least a portion of the tamper evident security connector. Even more preferably the diameter of the opening of the eyelet is from at least the width or diameter of a portion of the tamper evident security connector to be retained therein upon attachment and retention of the anchor portion with the engagement portion to twice that diameter or width. The diameter of the opening of the eyelet is less than 1.8 times that diameter or width. Preferably, the diameter of the opening of the eyelet is less than 1.6 times that diameter or width. More preferably, the diameter of the opening of the eyelet is less than 1.4 times that diameter or width. Most preferably, the diameter of the opening of the eyelet is less than 1.2 times that diameter or width.

In an embodiment the tamper evident security connector further includes an elongate portion, wherein the elongate portion has a first end which is connected to the body portion and a second end adjacent to the frangible portion.

Preferably, the elongate portion has a length of about one quarter to about one third of an outer perimeter of the lid.

Preferably, the elongate portion is flexible. This allows the elongate portion to be manipulated so that at least the anchor portion and the frangible portion can be threaded through the eyelet. More preferably the at least part of the elongate portion is frictionally retained by an opening of the eyelet.

Preferably, the elongate portion extends from the outer perimeter of the lid.

Preferably, the frangible portion is an region of reduced thickness that is coaxially aligned with a longitudinal axis of the elongate portion.

In an embodiment, the frangible portion is a region of reduced thickness of the tamper evident security connector.

In an embodiment, the anchor portion is a male connector, and the engagement portion includes a female connector.

Preferably, the female connector is a cavity that has a shape that corresponds to the anchor portion.

Preferably, the anchor portion has a tapered shape widening from a tip of the anchor portion to a base of the anchor portion, the tapered shape of the anchor portion configured to displace side walls of the cavity to permit passage of the anchor portion therethrough, the displaced side walls configured to close over the base of the anchor portion to prevent withdrawal of the anchor portion from the cavity.

Preferably, the anchor portion is configured for attachment and retention with the engagement portion through a ratchet mechanism.

In an embodiment, the anchor portion includes barbs to attach and retain the anchor portion within the engagement portion.

In preferred embodiments, the tamper evident security connector is not a collar that is frangibly attached to the body portion of the lid.

In another aspect of the invention there is provided a medical container including a lid as previously defined.

In yet a further aspect of the invention there is provided a medical container for use with the lid according to any one of the preceding claims.

The engagement portion may be part of the medical container, or may be mounted to the medical container, such as on a collar of the medical container.

In one embodiment the engagement portion is integrally formed with a body of the medical container. This may be desirable where the container is a disposable container. After the removing the lid to open the container the anchor portion will remain retained within the engagement portion. Preferably, the anchor portion is not removable or disconnectable from the engagement portion. This prevents re-use of the container. Thus, where it is desired that the container is a single use container that must be disposed of after opening, it can be advantageous to have engagement portion that is integrally formed with the container.

In an alternative embodiment the engagement portion is integrally formed on a collar that is separable from a body of the medical container. Preferably, the collar is separable from the body of the medical container only in the absence of a lid. That is, attachment of the lid to the container prevents removal of the collar. More preferably, to remove the collar, the lid first needs to be detached from the container. The detachment process causes the frangible portion of the tamper evident security connector to break. The lid can then be removed from the container, and the collar can be subsequently removed. A removable collar having the engagement portion integrally formed thereon may be advantageous where it is desired to provide a re-usable container.

Further aspects of the present invention and further embodiments of the aspects described in the preceding paragraphs will become apparent from the following description, given by way of example and with reference to the accompanying drawings.

### BRIEF DESCRIPTION OF THE SEVERAL VIEWS OF THE DRAWINGS

FIG. 1 is an illustration of a preferred embodiment of a container including a lid that has a tamper evident security connector in an unconnected state.

FIG. 2 is an illustration of a lid and security connector.

FIG. 3 is an illustration of a lid with a tamper evident security connector connected to a body of a container.

FIG. 4 shows Detail 1 of FIG. 3 illustrating the connection of the tamper evident security connector to the container.

FIG. 5 shows an alternate embodiment of a lid and collar.

FIG. 6 is a sectional view along lines B-B of FIG. 5.

FIG. 7 shows Detail 2 of FIG. 6 illustrating the connection of the tamper evident security connector to the collar.

### DETAILED DESCRIPTION

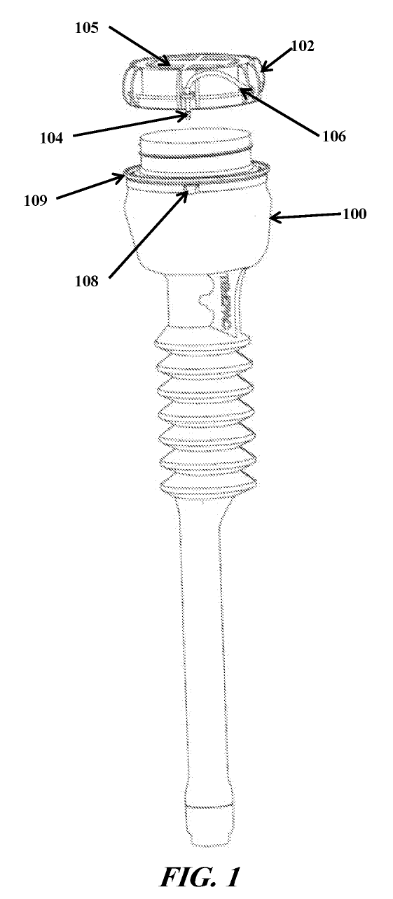

FIG. 1 is an illustration of a preferred embodiment of a container 100 including a lid 102 that has a tamper evident security connector 104 according to the present invention, wherein the tamper evident security connector 104 in its disconnected state.

In this embodiment the lid 102 is a screw cap lid. However, the skilled addressee will appreciate that other types of lids may be used.

In this embodiment, the container 100 and lid 102 combine to form an assembly that is typically used for sterilization and storage of sterilized medical instruments. Broadly, a medical instrument is placed within the container 100, and the lid 102 is then sealed to the container 100. The sealed container 100 may then be placed within an autoclave to sterilize the medical instrument stored within. Once sterilized, the medical instrument can be stored within the container 100 until required. The container 100 and lid 102 includes a tamper evident security connector 104 which indicates whether the container 100 has been opened, and thus the sterility of the medical instrument therein.

In order for the lid 102 to be suitable for use in an autoclave, the lid must be able to withstand the typical temperatures and pressures required within the autoclave during sterilization. Furthermore, in such cases, the lid 102 includes a vapor permeable sterility barrier 105 to allow the passage of vapor, preferably steam, into and out of the container 100. This permeable sterility barrier 105 is preferably a non-wettable vapor permeable sterility barrier 105. In such cases, it is also preferred that the non-wettable vapor permeable sterility barrier 105 is hydrophobic. A hydrophobic vapor sterility barrier 105 advantageously allows the passage of water and water vapor therethrough, but due to the hydrophobic nature prevents surfaces of the barrier from being wetted by water. As the barrier cannot be wetted, strikethrough does not occur. Strikethrough is a problem that occurs when a porous membrane (such as a permeable sterility barrier 105) becomes wetted. Wetting of the permeable sterility barrier 105 increases the risk of transfer of pathogens through the barrier 105, compromising sterility. In this specific embodiment, the vapor permeable sterility barrier includes a microporous membrane formed from a hydrophobic polymeric material. A range of different materials may be used, provided that those materials are able to withstand autoclave conditions. Suitable materials include polypropylene, polyesters and polytetrafluoroethylene.

The tamper evident security connector 104 is formed integrally with the lid 102. That is, the lid 102 and the tamper evident security connector 104 are unitary. Suitable methods for forming a lid 102 that includes an integrally formed tamper evident security connector 104 may be through injection molding. In this case, the lid 102 and the tamper evident security connector 104 are formed through an injection molding process from the same material.

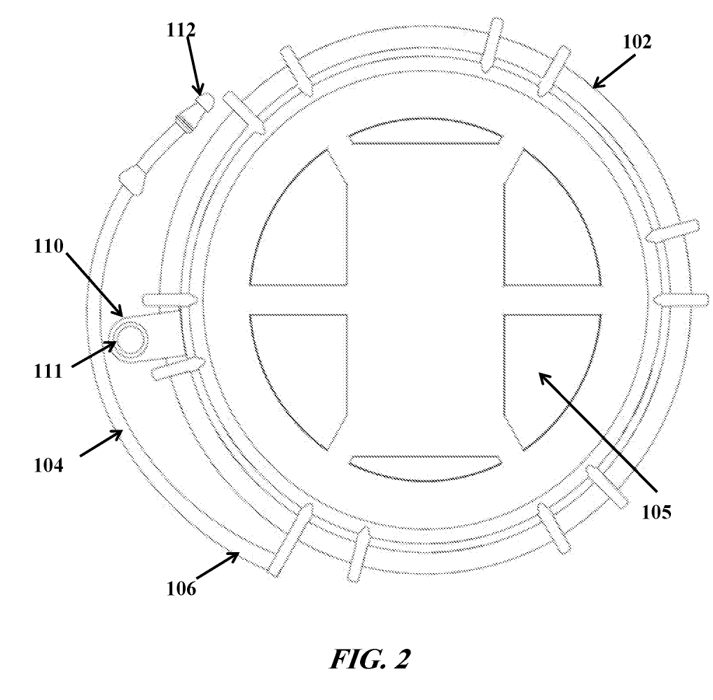

The lid 102 is shown in greater detail in FIG. 2. In this embodiment the lid 102 includes a tamper evident security connector 104 having an elongate portion 106 which extends from an outer perimeter of the lid 102. The skilled addressee will appreciate that the tamper evident security connector 104 may conveniently extend from other portions of the lid 102 depending on the design and nature of the lid. The elongate portion 106 has a degree of flexibility such that it can be manipulated for connection with an engagement portion 108 of the container 100.

The engagement portion 108 may be formed integrally with the container 100. That is the engagement portion 108 and the container are a unitary piece. Alternatively, the engagement portion 108 may instead be present on a collar 109 which is connected with the container 100. In such embodiments, collar 109 may be physically attached to the container 100 for example via some form of non-removable connection means or mechanism, through direct physical attachment (such as via welding or the like), or further the collar 109 may be glued or otherwise adhered to the container 100. For the purpose of the discussion in respect of FIGS. 1 to 4 below, the collar 109 is connected to the container 100 via one of the aforementioned means and is not removable. Therefore, this discussion also generally applies to a container in which the engagement portions 108 are integrally formed with the container 100. However, alternative embodiments in which the collar 109 is removable will also be discussed.

The elongate portion 106 extends from the outer perimeter of the lid 102 and is substantially aligned with the circumference of the lid. Again, the skilled addressee will appreciate that the nature of the alignment of the elongate portion 106 with respect to the outer perimeter of the lid 102 will depend on the size, shape, and physical characteristics of the lid 102. The elongate portion 106 is of sufficient length that it can engage with an engagement portion 108 of the container 100. In the present embodiment, the elongate portion 106 is approximately one third the length of the outer circumference of the lid 102.

In the presently illustrated embodiment, the container 100 includes one engagement portions 108. However, the skilled addressee will appreciate that the container may include more than one engagement portions, such as two engagement portions. This can be advantageous as it allows a user to connect the lid 102 to the container 100 without being concerned with the exact orientation of the lid 102. Instead, the tamper evident security connector 104 will naturally be in sufficiently close proximity to one of the two engagement portions that the tamper evident security connector 104 can be connected to at least one of the engagement portions Furthermore, in alternative embodiments, the container 100 may include multiple tamper evident security connectors corresponding with each of the engagement portions of the container. On connection of the lid to the container each of the tamper evident security connectors is in proximity to form a connection with a corresponding engagement portion of the container.

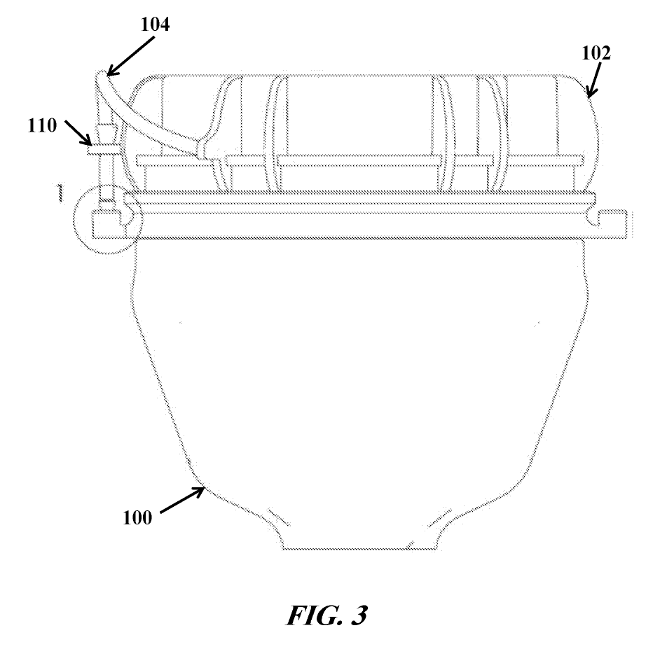

In the presently illustrated embodiment, particularly with reference to FIG. 2, the lid 102 also includes a locating element 110, which in this case is an eyelet. As best shown in FIG. 3, when the lid 102 is connected to the container 100, a hole of the eyelet 111 (see FIG. 2) is substantially coaxially aligned with the engagement portion 108 of the lid 102, such that the tamper evident security connector 104 may be threaded through the hole of the eyelet 110 to be connected with the engagement portion 108. Thus, on attachment of the lid 102, locating element or eyelet 110 is configured to locate with the engagement portion 108, to allow an anchor portion 112 to be easily inserted through the eyelet 110 and into the engagement portion 108. The engagement portion 108 retains the anchor portion 112 therein to lock the security connector 104 in place. Attempting to remove the security connector 104 will cause the security connector 104 to break, leaving the anchor portion 112 within the engagement portion 108. Thus for example, unscrewing the lid 102 to separate the lid 102 from the container 100 causes the security connector 104 to break, which indicates to the user that the container 100 has been accessed.

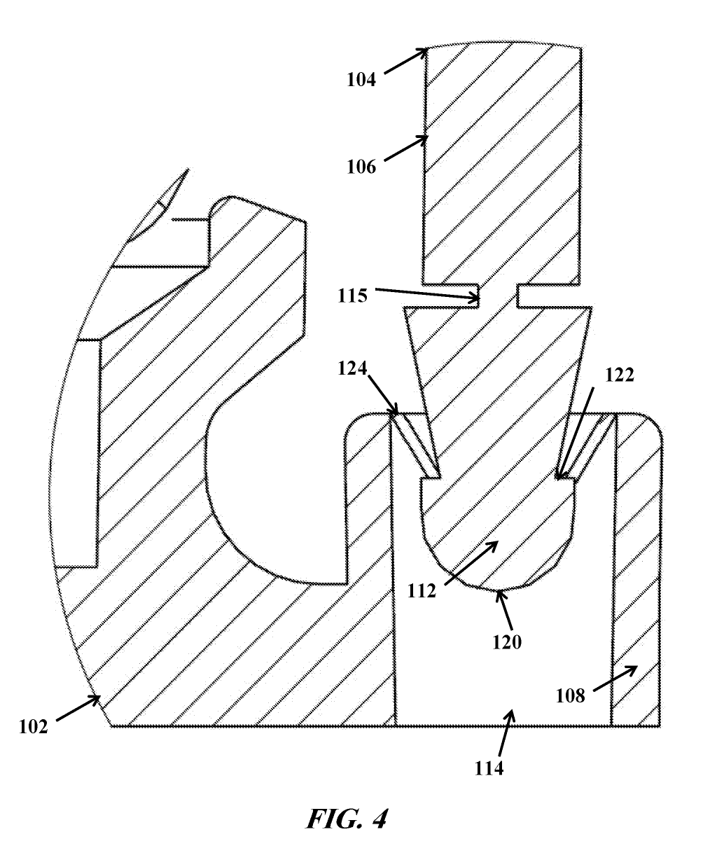

As discussed above, the tamper evident security connector 104 is connected to the engagement portion 108 of the container 100 via an anchor portion 112. FIG. 4 illustrates the end of the security connector 104 retained within the engagement portion 108 of the lid 102 after insertion of the anchor portion 112. The anchor portion 112 is shaped for engagement with a correspondingly shaped cavity 114 in the engagement portion 108. This correspondingly shaped cavity 114 is configured to receive at least the anchor potion 112 of the tamper evident security connector 104, to retain the anchor portion in place and to prevent its withdrawal after insertion.

The use of the security connector in the context of the container 100 and lid 102 will now be discussed. As previously discussed, FIG. 3, illustrates an embodiment in which the tamper evident security connector 104 has been connected with the engagement portion 108 of the container 100. As can be seen, the tamper evident security connector 104 is threaded through the hole of the eyelet 110 and the anchor portion 112 has been inserted into the engagement portion 108 of the container 100, where the anchor portion 112 is retained within the correspondingly shaped cavity 114 of the engagement portion 108. Once the lid 102 and container 100 have been connected in this manner, the lid 102 cannot be removed from the container 100 (for example by attempting to pull the anchor portion 112 from the correspondingly shaped cavity 114 or unthreading the lid 102 from the container 100) without breaking the tamper evident security connector 104 at a frangible portion 115 (see FIG. 4) of the tamper evident security connector 104.

FIG. 4 shows further detail of the connection between the anchor portion 112 of the tamper evident security connector 104 within the correspondingly shaped cavity 114 of the engagement portion 108. The correspondingly shaped cavity 114 is shaped to permit entry of the anchor portion 112 into the engagement portion 108 and to prevent withdrawal of the anchor portion 112 from the engagement portion 108 after insertion. A suitable type of connection may be interference connection in which the anchor portion 112 of the tamper evident security connector 104 is a male connector, and the correspondingly shaped cavity 114 of the engagement portion is a female connector.

In this particular embodiment, the anchor portion 112 has a narrow tip 120 which widens out to form a tapered head from the tip 120 of the anchor portion 112 to a base 122 of the anchor portion 112, wherein the base 122 of the anchor portion 112 adjoins the elongate portion 106 of the tamper evident security connector 104. The base 122 of the anchor portion 112 presents a flanged surface which engages with a corresponding flanged surface of the correspondingly shaped cavity 114 to prevent withdrawal. On insertion of the anchor portion 112 into the correspondingly shaped cavity 114, the tapered shape of the anchor portion 112 pushes narrowing side walls 124 of the correspondingly shaped cavity 114 apart permitting the anchor portion 112 to pass into the correspondingly shaped cavity 114. Once the anchor portion 112 is inserted into the correspondingly shaped cavity 114 the narrowing side walls 124 close against the anchor portion 112 such that the flanged surface of the correspondingly shaped cavity 114 closes over the flanged surface of the base 122 to hold the anchor portion 112 in position, and to prevent withdrawal of the anchor portion 112. Attempting to remove the anchor portion 112 from the correspondingly shaped cavity 114 causes the tamper evident security connector 104 to break at a frangible portion 115 of the tamper evident security connector 104. In this case, the frangible portion 115 is structurally weakened region of reduced thickness.

In alternative embodiments, the anchor portion may additionally include barbs which further act to retain the anchor portion within the correspondingly shaped cavity and to prevent withdrawal of the anchor portion from the correspondingly shaped cavity after insertion.

When a user desires to open the container 100 the user rotates the lid 102 to de-thread the lid 102 from the container. As the user rotates the lid 102, tension is applied across the frangible portion 115 of the tamper evident security connector 104. In particular, during rotation of the lid 102, an internal wall of the locating element, or in this embodiment eyelet 110 pushes against the tamper evident security connector 104 at a location that is near to, or adjacent, the frangible portion 115. This assists in concentrating the tension force at the frangible portion 115. The eyelet 110 also assists in breaking the frangible portion after only a small degree of rotation is applied to the lid 102. In the absence of the eyelet 110, the lid would undergo partial rotation, for example around a quarter turn of the lid 102, until the elongate portion 106 had been fully extended. This could potentially allow a lid of a container to be lifted or disconnected, without breaking the frangible portion, for example this may arise where a lid has only a short thread for engagement with a container.

In any event, once this tension force reaches a critical value, the tamper evident security connector 104 breaks at the frangible portion 115, leaving the anchor portion 112 in the correspondingly shaped cavity 114 of the engagement portion 108. The elongate portion is 106 is retained in place by the eyelet 110. That is, in the absence of the eyelet 110, the elongate portion would droop or jut-out becoming an encumbrance. Thus, the eyelet 110, retains the elongate portion 108 in position relative to the lid 102 through frictional engagement.

Once the tamper evident security connector 104 has broken, the tamper evident security connector 104 cannot be re-inserted into the engagement portion 108 as at least (i) the anchor portion 112 has been separated from the tamper evident security connector 104 and as such there is nothing to anchor the tamper evident security connector 104 into the engagement portion 108, and (ii) the anchor portion 112 has been retained within the tamper evident security connector 104, and this prevents insertion of a further such portion into the correspondingly shaped cavity 114 of the engagement portion 108.

Additionally, given the above, the skilled user can readily ascertain whether the container has been opened (or potentially tampered with) on a visual inspection of the state of the tamper evident security connector 104.

As discussed above, in some embodiments the collar 109 is separable from the container 100. In such embodiments, while the collar 109 may be removed when the lid 102 is not connected to the container 100, the collar 109 cannot be removed while the lid 102 is attached to the container 100. That is, in order to remove the collar 109, the lid 102 first needs to be detached from the container 100. This ensures, that even though the collar 109 is removable, the lid 102 first needs to be decoupled from the container 100, causing the tamper evident security connector 104 to break at the frangible portion 126.

The advantage of having a removable collar 109 is that the container 100 can be reused. That is, in certain embodiment where the engagement portion 108 are either integrally formed with the container 100 or reside on an irremovable collar 109, the container is generally not reusable as the anchor portion 112 of the tamper evident security connector 104 is left jammed within the correspondingly shaped cavity 114 of the engagement portions 108. Thus, in such embodiments the container must be disposed of after use. However, in embodiments which include a removable collar 109, after use, the removable collar 109 having the anchor portion 112 of the tamper evident security connector 104 retained within the correspondingly shaped cavity 114, can be removed from the container 100 and disposed of. A new collar 109 can then be applied and the container 100 re-used after appropriate treatment if required, such as a sterilization treatment.

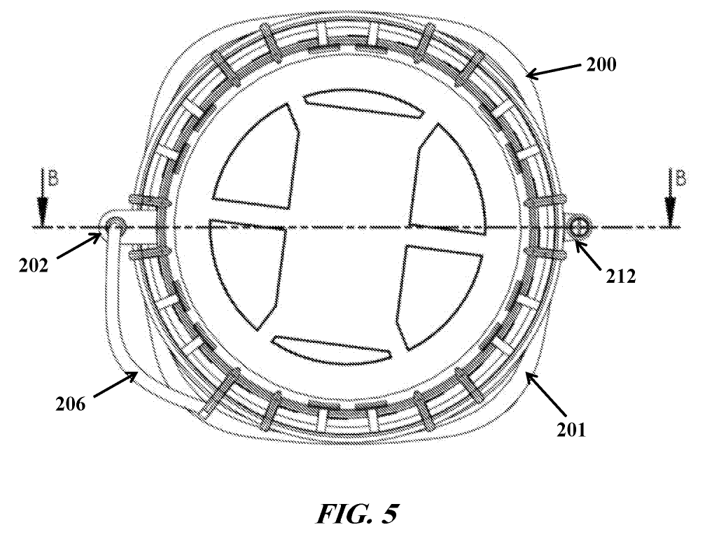

An alternative embodiment of a lid 200 connected to a collar 201 (for example, of a container) is illustrated in FIGS. 5, 6, and 7. In this embodiment, the lid 200 includes a locating element 202 and a security connector 206. The security connector 206 may optionally be passed through the locating element 202 to connect with one of two corresponding engagement portions (one of which is not shown, the other of which is 212) of the collar 201. The lid 200 and collar 201 are arranged such that at least one of the engagement portions are substantially aligned with the locating element 202.

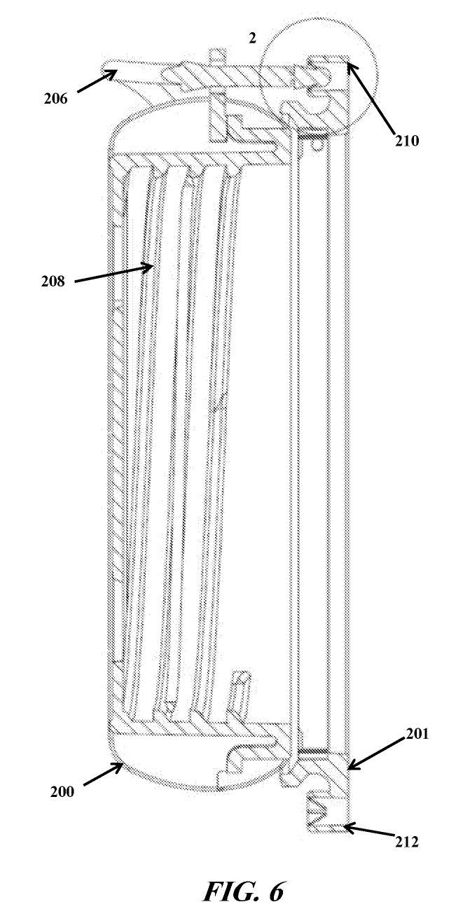

FIG. 6 provides a cross-sectional view along line B-B of the lid 200 and collar 201 shown in FIG. 5. As can be seen, the lid 200 includes a screw thread 208 for connection with a container (not shown). The collar 201 is also shown as including two engagement portions 210 and 212, wherein at least one of the engagement portions 210 or 212 is substantially aligned with the locating element 202 such that the security connector 206 can be passed through locating element 202 to be retained by one of engagement portions 210 or 212.

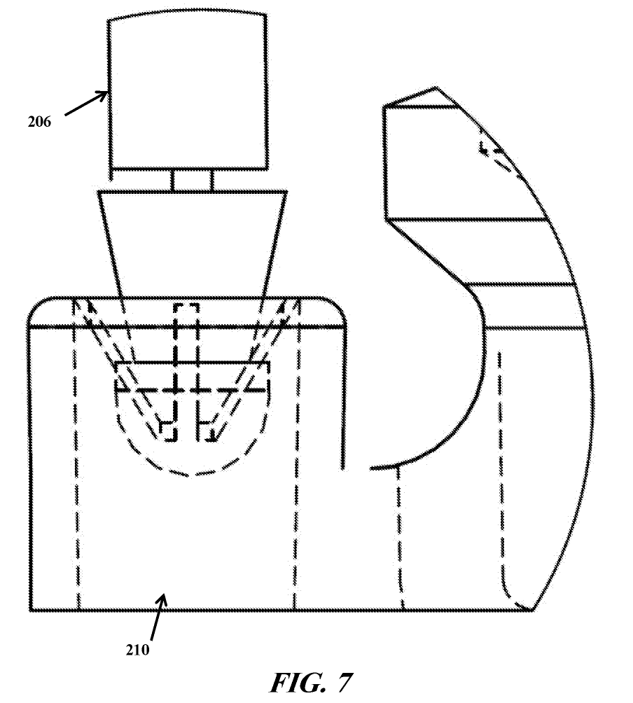

FIG. 7 shows Detail 2 of FIG. 6, illustrating the means of connection between the security connector 206 and the engagement portion 210. This interaction is similar to that described with reference to FIG. 4.

With reference to FIG. 6, the lid 200 and collar 201 may be connected to a container such that the collar 201 is retained in place around the neck of the container. Suitable methods are known to those skilled in the art, but for example, the collar 201 and container may together interact through a ratchet type arrangement that permits the collar 201 to be rotationally fitted with the lid 200 to the container. However, removal of the lid 200 from the container by rotating in the reverse direction (for example to dethread the lid 200 from the container) causes the ratchet arrangement to prevent the reverse rotation of the collar 201 with respect to the container. Thus, a force is applied to the security connector 206 which breaks the frangible portion as generally described previously.

It will be understood that the invention disclosed and defined in this specification extends to all alternative combinations of two or more of the individual features mentioned or evident from the text or drawings. All of these different combinations constitute various alternative aspects of the invention.

It will be understood that the invention disclosed and defined in this specification extends to all alternative combinations of two or more of the individual features mentioned or evident from the text or drawings. All of these different combinations constitute various alternative aspects of the invention.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A lid for a medical container, the lid including:
a body portion including a locating element,
a tamper evident security connector integrally formed with the body portion, the tamper evident security connector including:
an anchor portion, and
a frangible portion;
wherein the anchor portion is configured to attach to and be retained by an engagement portion fixed in at least one direction relative to the medical container, thereby positioning the frangible portion between the locating element and engagement portion, the frangible portion being adapted to break on relative movement between the lid and the medical container; and
wherein the lid is suitable for use in an autoclave, the lid being formed from a material or materials that are capable of withstanding a temperature of at least 140° C.

2. The lid of claim 1, wherein the locating element is configured to coaxially align with the engagement portion.

3. The lid of claim 1, wherein the locating element is configured to frictionally engage with the tamper evident security connector, and to substantially retain the tamper evident security connector in position relative to the body portion of the lid.

4. The lid claim 1, wherein the locating element is an eyelet configured to allow passage of the anchor portion therethrough to attach and be retained by the engagement portion of the medical container.

5. The lid claim 1, wherein the tamper evident security connector further includes a flexible elongate portion, wherein the elongate portion has a first end which is connected to the body portion and a second end adjacent to the frangible portion.

6. The lid of claim 5, wherein the elongate portion has a length of about one quarter to about one third of an outer perimeter of the lid, the outer perimeter of the lid being in a plane that is parallel with an opening of the container.

7. The lid of claim 5, wherein the elongate portion extends from the outer perimeter of the lid.

8. The lid of claim 5, wherein the frangible portion is a region of reduced thickness that is coaxially aligned with a longitudinal axis of the elongate portion.

9. The lid of claim 1, wherein the frangible portion is a region of reduced thickness of the tamper evident security connector.

10. The lid of claim 1, wherein the anchor portion is a male connector, and the engagement portion includes a female connector.

11. The lid of claim 10, wherein the female connector is a cavity that has a shape that corresponds to the anchor portion.

12. The lid of claim 11, wherein the anchor portion has a tapered shape widening from a tip of the anchor portion to a base of the anchor portion, the tapered shape of the anchor portion configured to displace side walls of the cavity to permit passage of the anchor portion therethrough, the displaced side walls configured to close over the base of the anchor portion to prevent withdrawal of the anchor portion from the cavity.

13. The lid of claim 1, wherein the anchor portion includes barbs to attach and retain the anchor portion within the engagement portion.

14. The lid of claim 1, wherein the lid further includes a permeable portion that allows transmission of a sterilization agent.

15. The lid of claim 14, wherein the permeable portion is a vapor permeable membrane,

16. A medical container including a lid according to claim 1.

17. (canceled)

18. The medical container of claim 16, wherein the engagement portion is integrally formed with a body of the medical container.

19. The medical container of claim 16, wherein the engagement portion is integrally formed on a collar that is separable from a body of the medical container.

20. The medical container of claim 19, wherein the engagement portion is fixed relative to the container.

21. The medical container of claim 20, wherein the container includes the engagement portion.
